(19) United States
(12) Patent Application Publication
(10) Pub. No.: US 2024/0296426 A1
(43) Pub. Date: Sep. 5, 2024

(54) SYSTEMS AND METHODS FOR FACILITATING DETERMINING FITNESS OF INDIVIDUALS FOR AN EMPLOYMENT POSITION

(71) Applicant: Ayan Yeleuov, Almaty (KZ)

(72) Inventor: Ayan Yeleuov, Almaty (KZ)

(21) Appl. No.: 18/662,829

(22) Filed: May 13, 2024

Related U.S. Application Data
(63) Continuation-in-part of application No. 17/809,205, filed on Jun. 27, 2022.

(51) Int. Cl. G06Q 10/1053; G06N 20/00; G09B 7/00

(52) U.S. Cl. CPC G06Q 10/1053; G06N 20/00; G09B 7/00

(57) ABSTRACT

The present invention introduces a method and system for assessing employment suitability using a communication device and a processing device equipped with a machine learning chatbot, enhanced by robust security measures. The process starts by collecting and analyzing personal data from individuals to generate preliminary scores. Customized tests are administered to verify these scores, with questions randomized when multiple individuals are involved to prevent cheating. Security protocols include IP address verification and environmental monitoring. The dynamic testing adjusts question difficulty based on responses. Test results are analyzed to produce verified scores, which calculate a human capital score for each individual, indicating their job suitability.

### FIELD OF DISCLOSURE

The present invention generally relates to the field of data processing and employment assessment technologies. More specifically, the present invention relates to systems and methods for evaluating the suitability of individuals for specific job roles, utilizing advanced data processing techniques to facilitate the determination of an individual's fitness for an employment position.

### BACKGROUND

The evaluation of individual fitness for employment positions is a critical concern in the modern workforce, where the field of data processing plays an essential technological role across various industries, businesses, and personal applications.

Existing methods for assessing the suitability of candidates for specific job roles are predominantly dependent on outdated tools such as resumes and self-assessment instruments. These traditional approaches often fail to capture the full spectrum of a candidate's capabilities and potential contributions to a role, leading to inefficiencies in the hiring process and potential mismatches in job placements.

Thus, there exists a significant need to advance the methodologies used to evaluate individual fitness for employment positions. This methodological advancement is crucial to address the inherent limitations of current systems and to better align candidate evaluations with the dynamic requirements of modern job roles.

The present invention seeks to address these challenges by introducing innovative features and enhanced problem-solving capabilities into the conventional systems and methods used for assessing employment suitability. The present invention aims to provide a more comprehensive, accurate, and efficient approach to determining the fitness of individuals for specific job roles, thereby improving the overall hiring process and outcomes for both employers and candidates.

### SUMMARY OF DISCLOSURE

This summary is provided to introduce a selection of concepts in a simplified form, that are further described below in the Detailed Description. This summary is not intended to identify key features or essential features of the claimed subject matter. Nor is this summary intended to be used to limit the claimed subject matter's scope.

The present disclosure provides a method for facilitating determining fitness of individuals for an employment position. The present invention incorporates the use of a communication device, a processing device equipped with a machine learning chatbot, and various security measures to ensure the integrity of the evaluation process.

The method and system begins by collecting personal data from individuals via their devices. This data is analyzed using the machine learning chatbot, which may include an Interactive Voice Response (IVR) module and a sentiment analyzer, to determine characteristics relevant to specific job positions. Based on this analysis, preliminary scores are generated for each individual.

Subsequent steps involve administering customized tests to verify these characteristics. The system retrieves test questions from a storage device, and questions are randomized if multiple individuals are involved. Security protocols include IP address verification, device control with individual consent, and environmental monitoring to detect any potential cheating during the testing phase.

The testing process is dynamic, with the difficulty of questions adjusted based on individual responses and the avoidance of question repetition. When multiple test takers are involved, questions can be randomized to prevent cheating and ensure the integrity of the test.

All responses are analyzed to generate test scores, which are then compared with initial scores (unverified scores) to produce verified scores. These verified scores are used to calculate a human capital score for each individual, indicating their suitability for the job position. Finally, the human capital score is transmitted to the individuals' devices, completing the evaluation process. This invention provides an efficient, secure, and automated method to assess employment suitability, leveraging the latest in machine learning and data processing technologies.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTIONS OF DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 is an illustration of an online platform consistent with various embodiments of the present disclosure.

FIG. 2 is a block diagram of a computing device for implementing the methods disclosed herein, in accordance with some embodiments.

FIG. 3 illustrates a flowchart of a method for receiving data in accordance with some embodiments.

FIG. 4 illustrates a flowchart of a method for identifying tests in accordance with some embodiments.

FIG. 5 illustrates a flowchart of a method for control and monitoring in accordance with some embodiments.

FIG. 6 illustrates a flowchart of a method for test questions in accordance with some embodiments.

FIG. 7 illustrates a flowchart of a method for generating human capital scores in accordance with some embodiments.

FIG. 8 illustrates a block diagram of a system for facilitating determining fitness of individuals for an employment position, in accordance with some embodiments.

FIG. 9 illustrates a block diagram of the system, in accordance with some embodiments.

FIG. 10 illustrates a block diagram of the system, in accordance with some embodiments.

FIG. 11 illustrates a block diagram of the system, in accordance with some embodiments.

FIG. 12 illustrates a block diagram of the system, in accordance with some embodiments.

FIG. 13 is a table illustrating attributes of individuals (candidates) for facilitating determining fitness of individuals for an employment position, in accordance with some embodiments.

FIG. 14 is a continuation of the table illustrating the attributes of the individuals (candidates) for facilitating the determining of the fitness of the individuals for the employment position, in accordance with some embodiments.

FIG. 15 is a table illustrating work experience scores of individuals (candidates) for facilitating determining fitness of individuals for an employment position, in accordance with some embodiments.

FIG. 16 is a table illustrating educational background scores of individuals (candidates) for facilitating determining fitness of individuals for an employment position, in accordance with some embodiments.

FIG. 17 is a table illustrating quotient scores of individuals (candidates) for facilitating determining fitness of individuals for an employment position, in accordance with some embodiments.

FIG. 18 is a table illustrating age multiplication factors of individuals (candidates) for facilitating determining fitness of individuals for an employment position, in accordance with some embodiments.

### DETAILED DESCRIPTION OF DISCLOSURE

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim limitation found herein and/or issuing here from that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the claims found herein and/or issuing here from. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of systems and methods for facilitating determining fitness of individuals for an employment position, embodiments of the present disclosure are not limited to use only in this context.

In general, the method disclosed herein may be performed by one or more computing devices. For example, in some embodiments, the method may be performed by a server computer in communication with one or more client devices over a communication network such as, for example, the Internet. In some other embodiments, the method may be performed by one or more of at least one server computer, at least one client device, at least one network device, at least one sensor and at least one actuator. Examples of the one or more client devices and/or the server computer may include, a desktop computer, a laptop computer, a tablet computer, a personal digital assistant, a portable electronic device, a wearable computer, a smart phone, an Internet of Things (IoT) device, a smart electrical appliance, a video game console, a rack server, a super-computer, a mainframe computer, mini-computer, micro-computer, a storage server, an application server (e.g. a mail server, a web server, a real-time communication server, an FTP server, a virtual server, a proxy server, a DNS server etc.), a quantum computer, and so on. Further, one or more client devices and/or the server computer may be configured for executing a software application such as, for example, but not limited to, an operating system (e.g. Windows, Mac OS, Unix, Linux, Android, etc.) in order to provide a user interface (e.g. GUI, touch-screen based interface, voice based interface, gesture based interface etc.) for use by the one or more users and/or a network interface for communicating with other devices over a communication network. Accordingly, the server computer may include a processing device configured for performing data processing tasks such as, for example, but not limited to, analyzing, identifying, determining, generating, transforming, calculating, computing, compressing, decompressing, encrypting, decrypting, scrambling, splitting, merging, interpolating, extrapolating, redacting, anonymizing, encoding and decoding. Further, the server computer may include a communication device configured for communicating with one or more external devices. The one or more external devices may include, for example, but are not limited to, a client device, a third party database, public database, a private database and so on. Further, the communication device may be configured for communicating with the one or more external devices over one or more communication channels. Further, the one or more communication channels may include a wireless communication channel and/or a wired communication channel. Accordingly, the communication device may be configured for performing one or more of transmitting and receiving of information in electronic form. Further, the server computer may include a storage device configured for performing data storage and/or data retrieval operations. In general, the storage device may be configured for providing reliable storage of digital information. Accordingly, in some embodiments, the storage device may be based on technologies such as, but not limited to, data compression, data backup, data redundancy, deduplication, error correction, data finger-printing, role based access control, and so on. Further, one or more steps of the method disclosed herein may be initiated, maintained, controlled and/or terminated based on a control input received from one or more devices operated by one or more users such as, for example, but not limited to, an end user, an admin, a service provider, a service consumer, an agent, a broker and a representative thereof. Further, the user as defined herein may refer to a human, an animal or an artificially intelligent being in any state of existence, unless stated otherwise, elsewhere in the present disclosure. Further, in some embodiments, the one or more users may be required to successfully perform authentication in order for the control input to be effective. In general, a user of the one or more users may perform authentication based on the possession of a secret human readable secret data (e.g. username, password, passphrase, PIN, secret question, secret answer etc.) and/or possession of a machine readable secret data (e.g. encryption key, decryption key, bar codes, etc.) and/or or possession of one or more embodied characteristics unique to the user (e.g. biometric variables such as, but not limited to, fingerprint, palm-print, voice characteristics, behavioral characteristics, facial features, iris pattern, heart rate variability, evoked potentials, brain waves, and so on) and/or possession of a unique device (e.g. a device with a unique physical and/or chemical and/or biological characteristic, a hardware device with a unique serial number, a network device with a unique IP/MAC address, a telephone with a unique phone number, a smartcard with an authentication token stored thereupon, etc.). Accordingly, the one or more steps of the method may include communicating (e.g. transmitting and/or receiving) with one or more sensor devices and/or one or more actuators in order to perform authentication. For example, the one or more steps may include receiving, using the communication device, the secret human readable data from an input device such as, for example, a keyboard, a keypad, a touch-screen, a microphone, a camera and so on. Likewise, the one or more steps may include receiving, using the communication device, the one or more embodied characteristics from one or more biometric sensors.

Further, one or more steps of the method may be automatically initiated, maintained and/or terminated based on one or more predefined conditions. In an instance, the one or more predefined conditions may be based on one or more contextual variables. In general, the one or more contextual variables may represent a condition relevant to the performance of the one or more steps of the method. The one or more contextual variables may include, for example, but are not limited to, location, time, identity of a user associated with a device (e.g. the server computer, a client device etc.) corresponding to the performance of the one or more steps, environmental variables (e.g. temperature, humidity, pressure, wind speed, lighting, sound, etc.) associated with a device corresponding to the performance of the one or more steps, physical state and/or physiological state and/or psychological state of the user, physical state (e.g. motion, direction of motion, orientation, speed, velocity, acceleration, trajectory, etc.) of the device corresponding to the performance of the one or more steps and/or semantic content of data associated with the one or more users. Accordingly, the one or more steps may include communicating with one or more sensors and/or one or more actuators associated with the one or more contextual variables. For example, the one or more sensors may include, but are not limited to, a timing device (e.g. a real-time clock), a location sensor (e.g. a GPS receiver, a GLONASS receiver, an indoor location sensor etc.), a biometric sensor (e.g. a fingerprint sensor), an environmental variable sensor (e.g. temperature sensor, humidity sensor, pressure sensor, etc.) and a device state sensor (e.g. a power sensor, a voltage/current sensor, a switch-state sensor, a usage sensor, etc. associated with the device corresponding to performance of the or more steps). Further, the one or more steps of the method may be performed one or more number of times. Additionally, the one or more steps may be performed in any order other than as exemplarily disclosed herein, unless explicitly stated otherwise, elsewhere in the present disclosure. Further, two or more steps of the one or more steps may, in some embodiments, be simultaneously performed, at least in part. Further, in some embodiments, there may be one or more time gaps between performance of any two steps of the one or more steps.

Further, in some embodiments, the one or more predefined conditions may be specified by the one or more users. Accordingly, the one or more steps may include receiving, using the communication device, the one or more predefined conditions from one or more and devices operated by the one or more users. Further, the one or more predefined conditions may be stored in the storage device. Alternatively, and/or additionally, in some embodiments, the one or more predefined conditions may be automatically determined, using the processing device, based on historical data corresponding to performance of the one or more steps. For example, the historical data may be collected, using the storage device, from a plurality of instances of performance of the method. Such historical data may include performance actions (e.g. initiating, maintaining, interrupting, terminating, etc.) of the one or more steps and/or the one or more contextual variables associated therewith. Further, machine learning may be performed on the historical data in order to determine the one or more predefined conditions. For instance, machine learning on the historical data may determine a correlation between one or more contextual variables and performance of the one or more steps of the method. Accordingly, the one or more predefined conditions may be generated, using the processing device, based on the correlation.

Further, one or more steps of the method may be performed at one or more spatial locations. For instance, the method may be performed by a plurality of devices interconnected through a communication network. Accordingly, in an example, one or more steps of the method may be performed by a server computer. Similarly, one or more steps of the method may be performed by a client computer. Likewise, one or more steps of the method may be performed by an intermediate entity such as, for example, a proxy server. For instance, one or more steps of the method may be performed in a distributed fashion across the plurality of devices in order to meet one or more objectives. For example, one objective may be to provide load balancing between two or more devices. Another objective may be to restrict a location of one or more of an input data, an output data and any intermediate data therebetween corresponding to one or more steps of the method. For example, in a client-server environment, sensitive data corresponding to a user may not be allowed to be transmitted to the server computer. Accordingly, one or more steps of the method operating on the sensitive data and/or a derivative thereof may be performed at the client device.

### Overview:

The present disclosure describes systems and methods for facilitating determining fitness of individuals for an employment position.

Further, the present disclosure describes a process, where “One si [symbol] e fits All” formula is used to determine job applicants' “Personal Human Capital” score, which is used to determine the applicant's fitness to a position in question. The disclosed system eliminates reliance on resume and/or other self-appraisal tools from job/candidate search, and is as such is an introduction of “Merit based system” based on actual skills applicants possess into the Hiring Process.

The process is used in a professional network platform with all functionalities of social-professional network, including but not limited to profile creation, networking, messaging, personal news-activity board, ability to post information, message, pic with the caption, ability react to messages, etc. and creation of job listings, company profile, etc.

Further, the present disclosure describes a Novel filter system that is based on Personal Human Capital score. It has been stated that ‘Human Capital is considered “intangible”. However, Human Capital can be quantified and it is tangible if proper mathematical modeling is applied.

In order to calculate Personal Human Capital score, computer implemented mathematical modeling is applied, using verifiable personal background data, such as working experience, volunteering experience, educational background, academic standing (GPA, number of publications, etc.), different measurable quotients (IQ, EQ, AQ, etc.), measurable skills and other relevant data from the personal background.

The formula is constantly evolving and changing to adjust to the current market environment, in other words, changes to the formula are to be applied when it will be determined that certain background data either lost or gained relevance factor.

The platform will ask candidates/users to identify their hard skill level, i.e. for each skill the user picks to associate with the candidate's profile, the platform will ask the user to assign a value from 1 to 10 (1-beginner, 10-expert). After that “unverified score” is generated for each skill. However, this unverified skill value will be later verified by an online test and will either be confirmed or will replace (supersede) the unverified score.

The “certified” skill verification test is performed continuously, using online test software algorithms and biometrical software, which will request validation after each question or randomly during the question. Question and answer parts of the test are timed, and candidates will have limited time to read/answer questions, to avoid “google” search for an answer, thus invalidating cheating techniques. Moreover, the question itself will not be available for re-reading during the answer part of the test, i.e. each task will consist of a separate question and answer parts and during the answer part of the task, the question will not be available for review to avoid “type in google” search for cheating purposes. In order to avoid “sit-in” cheating, where multiple candidates take tests in one room and share their answers vocally, questions and answer options are random and is picked from a database of possible questions/answers randomly, and will have a random set of N answers, where only 1 answer is correct, and 1 incorrect, whereas other answers will hold partial credit. Furthermore, the correct answer will have multiple wording versions, thus even the version of a correct answer for each question is random. Before the test will commence, the test taker is prompted to allow the platform to take control over the test taker's webcam, 360-degree Camera, or any other wide-angle camera that is capable to monitor surroundings and microphone, that can identify if candidates are receiving any vocal guidance from a phone, another person, etc.

Test takers are requested to isolate themselves in a quiet room during test time. The platform will monitor sound changes around test takers' area and will perform random and scheduled biometric verifications, if the platform will detect unusual sound activity or biometric verification will fail N number of times, the video and audio feed from test takers' computers are elevated to test administrators/monitors who will be able to monitor test takers' test attempt.

If cheating has been detected, the administrator/monitor will record evidence and test-takers will forfeit their attempt.

Finally, to avoid multiple candidates sitting in one room during the test, thus possibly cheating, the platform will verify candidates' IP addresses and prevent candidates from taking the test from one location. In addition, the platform will employ VPN detection techniques, and will not allow candidates to use VPN software during the test.
It is currently not possible to measure soft skills that candidates may possess, since there weren't any tests developed to measure soft skills, such as “team player”, “creative thinking”, “customer relations” etc. And as such, soft skills is measured using other data that the candidate provides, that is related to soft skills in questions.
Human capital is defined in the Oxford English Dictionary as “the skills the labor force possesses and is regarded as a resource or asset.” It encompasses the notion that there are investments in people (e.g., education, training, health) and that these investments increase an individual's productivity.
The term human capital refers to the economic value of a worker's experience and skills. Human capital includes assets like education, training, intelligence, skills, health, and other things employers value such as loyalty and punctuality. As such, it is an intangible asset or quality that isn't (and can't be) listed on a company's balance sheet. Human capital is perceived to increase productivity and thus profitability.
Further, the process may include registration steps, test-taking steps, etc:
In, the registration steps, the Applicant registers in the platform and provide necessary and verifiable background data, required to calculate Personal Human Capital, such as education level, major/minor, GPA, date of birth, work experience, all certificates/test scores, etc. that they have taken previously (IELTS, TOEFL, GRE, GMAT, etc.). All tests taken previously should be scored and should be from a recognized institution.
Moreover, the applicant (individuals) indicates a set of hard and soft skills the applicant perceives to possess.
Data required for the “One si [symbol] e fits All” formula to evaluate personal Human Capital, is variable, depending on current market demands and Human Capital Theory. Moreover, the “One si [symbol] e fits All” formula is ever-evolving to accommodate market demands and new developments in Human Capital theory.
In order to proceed applicant will have to provide a biometrical id to the platform and support all verifiable background data by uploading scans of the Diploma, official transcripts, certificates of employment, passport, etc.

Once a profile is created, the applicant is prompted to verify skills they possess, by taking a certified online test that will assign a score to each skill, which the applicant indicated during profile creation (Hard and soft skills tests, variation of quotients-IQ, EQ, AQ, VQ, etc. tests, certain psychological tests, etc.)

Once all data required for personal Human Capital calculation is available, the platform will calculate personal Human Capital which is used to assess the candidate's fitness to position in question.

In, the test-taking steps, During the tests session, the applicant is asked to adhere to a number of test rules including, but not limited to: the applicant is required to isolate for test duration in a quiet room, no mobile phones should be present on-premises, the candidate is required to sit in one position facing camera, to avoid discrepancies during random biometrical verification processes, etc.

Before the test will commence, applicants will have to digitally sign the agreement, confirming their understanding and adherence to test rules.

The platform will verify the applicant's id using biometrical verification and will request the applicant to provide access to the applicant's PC control over the video camera and microphone. A video camera will have to be of certain specifications, that will provide the platform with an ability to monitor the environment, i.e. camera has to be either wide-angle, 360-degrees, or rotating.

Before the test will commence, the camera allows the platform to map out the applicant's surroundings.

During the test camera will monitor surroundings, the microphone will monitor background noises. If discrepancy is noticed (unmapped or moving objects, irregular noises around test taker's area, or random biometrical verification test will fail more than N times), then the applicant is flagged for possible cheating attempt and microphone, camera feedback is elevated to Monitor, who will then closely monitor applicant's test attempt and will either determine if a cheating attempt is ongoing or will remove the flag and let applicant continue the test.

If the applicant will break the test's rules, the applicant will forfeit the test attempt and is flagged, during the next attempt flagged applicant is closely monitored.

Moreover, the platform will utilize additional steps to prevent cheating attempts including, but not limited to VPN tracker, blocking access to search engines and browsers, etc.

### Human Capital Calculation Algorithm

The algorithm of calculating Human capital is based on Human Capital definition:
The term human capital refers to the economic value of a worker's experience and skills. Human capital includes assets like education, training, intelligence, skills, health, and other things employers value such as loyalty and punctuality. As such, it is an intangible asset or quality that isn't (and can't be) listed on a company's balance sheet. Human capital is perceived to increase productivity and thus profitability.
Further, the algorithm may include the most desirable traits that individuals possess including, but not limited to: Hard skills, Soft skills, Education, Age, Health, Intelligence (IQ), Emotional Intelligence (EQ), Resilience (AQ), Creativity (CQ).
Important: Formula may change over time when new quotients that define desired characteristics individual may possess might be developed and/or when the market will change preferences in human capital characteristics and certain traits will lose relevance factor, whilst other traits will gain.
One si [symbol] e fits ALL formula:

(X*(SQ M.F).+Y*(Academic M.F.))*(IQ M.F.)*(EQ M.F.)*(AQ M.F.)*(Age M.F.)*(Health M.F.)

$$X = X 1 + X 2 = Skills average$$

$$X 1 = Soft skills average$$

$$X 2 = Hard skills average$$

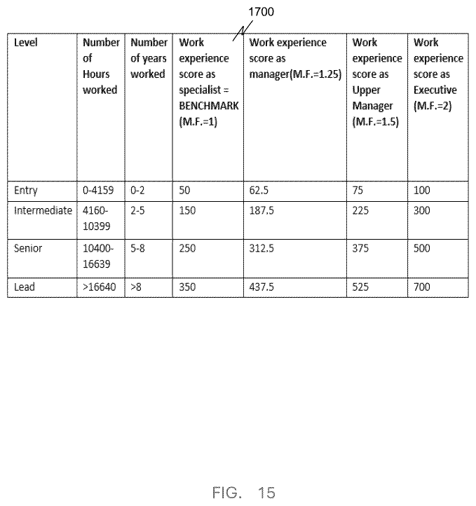

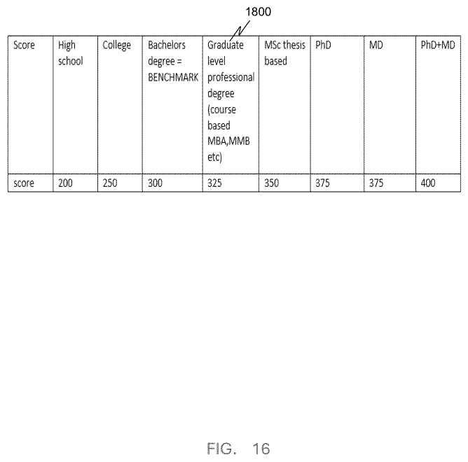

M.F.=multiplication factor, might vary between 0 to 1, where 1 is average (Health M.F.) or max possible (Academic M.F.), in some instances, it might reach 1.5 if individual scores exceptionally on certain quotients.
Academic M.F.=Academic multiplication factor derived from GPA over the last 2 years of higher education institution divided by maximum GPA that can be earned (i.e. If individual scores 3.5 GPA in the last 2 years of university out of 4.0 maximum GPA, then Academic M.F.=3.5/4=0.875)
SQ M.F=Skill Quotient multiplication factor and is calculated by dividing the number of skills candidate possess over the number of skills required for the position in question. i.e. if the candidate claimed and was tested on 5 skills, but only 4 of these skills match skills required for the position in question, SQ M.F.=4/5=0.8
IQ M.F.=Intellectual Quotient multiplication factor, derived from IQ score range, where the range is divided into categories: High, Above Average, Average, Below Average, and Low.
EQ M.F=Emotional Quotient multiplication factor, derived from EQ score, where the range is divided into categories: High, Above Average, Average, Below Average, and Low.
AQ M.F=Adversity Quotient multiplication factor, derived from AQ score, where the range is divided into categories: High, Above Average, Average, Below Average, and Low.
Age M.F.=Age multiplication factor, derived from individual's age following the same rule as stated previously in Academic M.F., i.e. individual's age divided by legal retirement age. This decision will lie on the employer, who has to determine if it is legally possible to perform such filters, if not then Age M.F. will remain as 1.
Health M.F.=Health multiplication factor, derived from individual's health, based on whether a person is healthy, thus multiplication factor is 1 or has a disability, where multiplication factor is as per employer's decision (i.e. if the job in question will demand physical activities, where disability is a limitation, such as construction worker, then Health M.F. might vary as per employer's decision from 0 to 1.5). This decision will lie on the employer, who has to determine if it is legally possible to perform such filters, if not then Health M.F. will remain as 1.
The philosophy behind the “One si [symbol] e fits ALL formula” is based on Human Capital is made up of educational background and experience. Thus, (X+Y)=100% Employers will adjust this equation as per their own requirement, i.e. by default in this equation, experience vs educational background weight in as such: Experience (X)=60%, whilst Educational background=40%. On the other hand, employers might use a different scenario, where X=70% and Y=30%, or X=50 and Y=50%, etc.
Further, the disclosed system may follow the assumption that experience is just a way to determine if an individual possesses the skills required to perform job related tasks. Moreover, skills are made up of 2 skill types: Hard and Soft. Thus, X consists of X1 and X2. Furthermore, the employer will have the ability to adjust which skills they prefer, hard or soft. By default, both types of skills is evenly weighted in the formula. However, employers will have an opportunity to further adjust this option. I.e. if the employer chooses default setting between experience and educational background or X=60% and Y=40, however, employer further prefers hard skills more than soft skills, X1=70%, X2=30%, then the formula is adjusted as such X1=0.7*Average hard skill score, whilst X2=0.3*Average soft skill score. To sum it up, the formula, per this scenario is adjusted as: ((0.7*Average hard skill score+0.3*Average soft skill score)*0.6+Y*0.4*(Academic M.F.))*(IQ M.F.)*(EQ M.F.)*(AQ M.F.)*(CQ M.F.)*(Age M.F.)*(Health M.F.) Further, Human Capital score is scored between 0 to 1000, with 1000 points awarded to a person aged below 40 y.o. with a Bachelors degree, who worked as a specialist for over 8 years, scoring max points on hard skill tests, where a list of candidate's hard and soft skills match 100% to those desired by Employer and averaging on all Quotients, this set is considered as BENCHMARK. Anything above 1000 is considered as an extra effort and will indicate exceptional candidates.
As was stated previously Hard skills average is calculated from the results of continuous online tests on hard skills that candidates indicate in their profile, with a maximum of 350 points per skill. The average is calculated, as usual, SumTotal of all skills points divided by a number of skills, thus maximum skill average is 350 points.
On the other hand, Soft skills, which are impossible to evaluate via online testing (i.e teamwork, customer relations, communicability, attention to details, creativity, etc.) is derived from working experience candidates indicate, using a number of hours candidate previously worked either full time or part-time, including volunteering experience.
Note: Each position candidate held while previously working is associated with soft skills that the candidate had to utilize to perform job related tasks. I.e. if the candidate worked as a store sales clerk, then soft skills such as communicability, teamwork, customer relations can be claimed and calculated towards Human Capital, whilst attention to details, creativity, or thinking “outside the box” might not.
In summary, “One si [symbol] e fits All” will look like this: (X1*(SQ M.F)+X2*(SQ M.F.)+Y*(Academic M.F.))*(IQ M.F.)*(EQ M.F.)*(AQ M.F.)*(CQ M.F.)*(Age M.F.)*(Health M.F.)=Personal Human Capital, max 1000 as per Benchmark, anything above is considered as an extra effort.
“One si [symbol] e fits All”′ formula utilization example in Human Capital calculation process: In a hypothetical example, where Employer choosing candidates' from the pool for a certain position. Employer adjusted preferences to 70% work experience and 30% educational background, whilst evenly preferring Hard and Soft skills (i.e 35% too hard skills and 35% too soft skills). In other words, soft skills will have a max of 350 points, hard skills max 350 points, and education max 300 points, as per Benchmark. Moreover, the Employer has a quota on people with disabilities and has adjusted disability as a preference (health M.F. for people with disability adjusted to 1.5). Candidates' attributes are summed up in the table 1500 as shown in FIGS. 15 and 16.
NOTE: All data tables are subject to change, whenever applicable. I.e. all quotients, work experience (soft skills), age and health multiplication coefficients are not permanent and are subject to change. “One si [symbol] e fits All” formula is ever evolving and will undergo change as per market demand and/or Human Capital theory changes. All benchmarks are subject to change.
Further, the present disclosure describes a process that will determine the personal Human Capital of applicants and will use it as a filtering mechanism to hone in on the most qualified applicants. Human Capital does not claim predictive mechanisms of any sort. Human capital is calculated as is, without any further extrapolation on candidate's performance, the disclosed system just assign Human Capital score that candidate possess de facto, without predicting how the applicant will perform work related duties or how well applicant will fit within an organization.

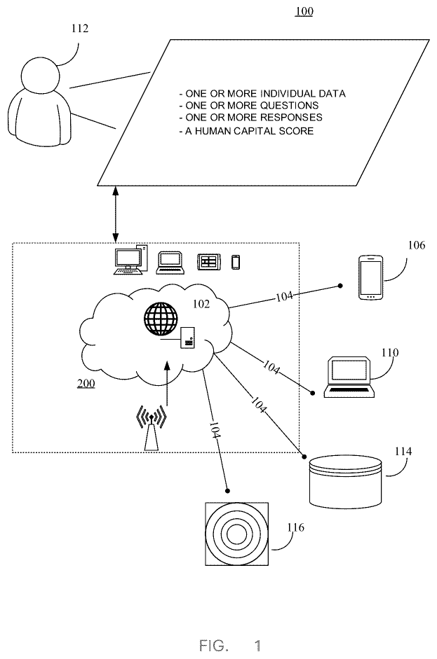

FIG. 1 is an illustration of an online platform 100 consistent with various embodiments of the present disclosure. By way of non-limiting example, the online platform 100 to enable facilitating determining fitness of individuals for an employment position may be hosted on a centralized server 102, such as, for example, a cloud computing service. The centralized server 102 may communicate with other network entities, such as, for example, a mobile device 106 (such as a smartphone, a laptop, a tablet computer etc.), other electronic devices 110 (such as desktop computers, server computers etc.), databases 114, and sensors 116 over a communication network 104, such as, but not limited to, the Internet. Further, users of the online platform 100 may include relevant parties such as, but not limited to, end-users, administrators, service providers, service consumers and so on. Accordingly, in some instances, electronic devices operated by the one or more relevant parties may be in communication with the platform.

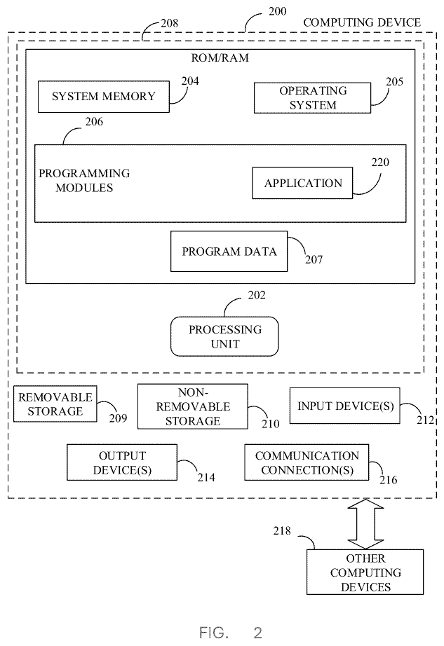

A user 112, such as the one or more relevant parties, may access online platform 100 through a web based software application or browser. The web based software application may be embodied as, for example, but not be limited to, a website, a web application, a desktop application, and a mobile application compatible with a computing device 200.
With reference to FIG. 2, a system consistent with an embodiment of the disclosure may include a computing device or cloud service, such as computing device 200. In a basic configuration, computing device 200 may include at least one processing unit 202 and a system memory 204. Depending on the configuration and type of computing device, system memory 204 may comprise, but is not limited to, volatile (e.g. random-access memory (RAM)), non-volatile (e.g. read-only memory (ROM)), flash memory, or any combination. System memory 204 may include operating system 205, one or more programming modules 206, and may include a program data 207. Operating system 205, for example, may be suitable for controlling computing device 200's operation. In one embodiment, programming modules 206 may include image-processing module, machine learning module. Furthermore, embodiments of the disclosure may be practiced in conjunction with a graphics library, other operating systems, or any other application program and is not limited to any particular application or system. This basic configuration is illustrated in FIG. 2 by those components within a dashed line 208. Computing device 200 may have additional features or functionality. For example, computing device 200 may also include additional data storage devices (removable and/or non-removable) such as, for example, magnetic disks, optical disks, or tape. Such additional storage is illustrated in FIG. 2 by a removable storage 209 and a non-removable storage 210. Computer storage media may include volatile and non-volatile, removable and non-removable media implemented in any method or technology for storage of information, such as computer-readable instructions, data structures, program modules, or other data. System memory 204, removable storage 209, and non-removable storage 210 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 200. Any such computer storage media may be part of device 200. Computing device 200 may also have input device(s) 212 such as a keyboard, a mouse, a pen, a sound input device, a touch input device, a location sensor, a camera, a biometric sensor, etc. Output device(s) 214 such as a display, speakers, a printer, etc. may also be included. The aforementioned devices are examples and others may be used.

Computing device 200 may also contain a communication connection 216 that may allow device 200 to communicate with other computing devices 218, such as over a network in a distributed computing environment, for example, an intranet or the Internet. Communication connection 216 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both storage media and communication media.

As stated above, a number of program modules and data files may be stored in system memory 204, including operating system 205. While executing on processing unit 202, programming modules 206 (e.g., application 220 such as a media player) may perform processes including, for example, one or more stages of methods, algorithms, systems, applications, servers, databases as described above. The aforementioned process is an example, and processing unit 202 may perform other processes. Other programming modules that may be used in accordance with embodiments of the present disclosure may include machine learning applications.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, general purpose graphics processor-based systems, multiprocessor systems, microprocessor-based or programmable consumer electronics, application specific integrated circuit-based electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems. Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory. Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid state storage (e.g., USB drive), or a CD-ROM, a carrier wave from the Internet, or other forms of RAM or ROM. Further, the disclosed methods' stages may be modified in any manner, including by reordering stages and/or inserting or deleting stages, without departing from the disclosure.

### Machine Learning Chatbot

The present invention includes a machine learning chatbot that serves as an interactive examiner. The chatbot uses natural language processing (NLP) to dynamically generate and pose both multiple-choice and written questions based on the candidate's performance and profile. The chatbot scores responses, monitors the examination environment for potential cheating, and adapts the question difficulty and depth accordingly. The chatbot is also capable of conducting additional evaluations as required, such as skills tests and quotient evaluations, using advanced NLP to analyze and score responses in real-time. It learns from accumulated test data to continually improve its algorithms.

The chatbot is equipped with AI-driven algorithms to monitor the test environment, analyzing video and audio inputs to detect potential cheating or discrepancies, ensuring the integrity and fairness of the testing process, especially in remote environments.

In some embodiments, the machine learning chatbot may used to determine or analize soft skills of individuals, wherein the machine learning chatbot may include an Interactive Voice Response (IVR) module configured to process inputs the user or individuals, wherein the IVR module may include: a) a processor configured to process digits, voice, and text data received from the user; and b) a sentiment analyzer coupled to the processor, configured to analyze prosodic data from the voice data, wherein the prosodic data includes at least one of intonation, tone, stress, and rhythm, to determine an emotional state of the user or individuals using the present invention.

The present invention also employs machine learning algorithms to analyze candidate profiles, identifying patterns and correlations that predict successful job performance. These algorithms adaptively refine the set of job-relevant characteristics in response to new data and evolving market demands, ensuring the criteria for evaluating candidates remain accurate and relevant.

In the realm of machine-learning evaluation, in some embodiments of the present invention, historical data and third-party data can be leveraged to develop and refine the machine learning models (chatbots) used for assessment. However, the volume of this historical and third-party data can often be substantially greater than that of the real-time data stream, which can lead to slower processing of the historical data. Therefore, in some implementations, the real-time processing pathway may exclude historical and third-party data as inputs. In other implementations, while historical and third-party data are incorporated, the bulk of the data utilized for real-time evaluation still primarily consists of data from current, incoming events.

In one embodiment, the present invention offers a system and method that encompasses the following steps:

### Receiving Data

Data Reception: The present invention receives individual data associated with one or more individuals from various devices using a communication device.
Analysis: This data is analyzed by a processing device and a machine learning chatbot to determine characteristics relevant to an employment position.
Characteristics Determination: Based on the analysis, certain characteristics of the individuals are identified.
Score Generation: Unverified scores are generated based on the individual data and the determined characteristics.

### Identifying Tests

Test Identification: The present invention identifies tests to verify the determined characteristics using the processing device.
Question Retrieval: Questions related to these tests are retrieved from a storage device.
Question Randomization: If the evaluation involves multiple persons, the questions are randomized.

### Control and Monitoring

Security Measures: The present invention verifies IP addresses, detects VPN usage, and blocks access to search engines and browsers to maintain test integrity.
Device Control: Individuals are prompted to grant control over their devices, allowing the system to take over cameras and microphones to monitor surroundings.
Environmental Mapping and Monitoring: The surroundings are mapped and monitored for sound and visual variations. Biometric verifications are conducted, and upon detecting anomalies or failures, warnings are issued and feeds are transferred to an authorized person for direct monitoring.
Cheating Detection: Evidence of cheating is recorded, and communication with involved individuals can be discontinued.

### Test Questions

Question Transmission and Display: Questions are transmitted to and displayed on individual devices, then removed after a set time.
Response Reception and Analysis: Responses are received within a predetermined time, analyzed, and used to generate subsequent questions, which are adjusted in difficulty by the machine learning chatbot.
Additional Questions: Additional questions specific to the employment position are also posed and managed in the same manner.
Feedback for Chatbot Training: Identical questions are identified and used to provide feedback for training the machine learning chatbot.

### Generating Human Capital Scores

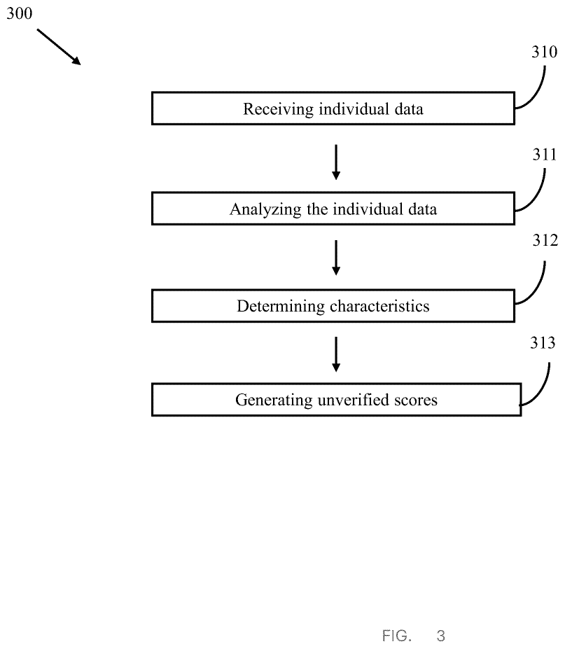

Score Comparison and Verification: Unverified scores are compared with test scores to generate verified scores.
Human Capital Score Calculation: A human capital score is calculated using a specific algorithm, indicating the suitability of the individuals for the employment position.
Score Transmission and Storage: This score is transmitted back to the individuals' devices and stored in the storage device for record-keeping.
   FIG. 3 illustrates a flowchart of a method 300 for facilitating determining fitness of individuals for an employment position, in accordance with some embodiments. Further, the employment position may include a job, a position in an organization, etc.
   Accordingly, the method 300 may include steps for Receiving Data In one embodiment, the method 300 may include steps of
a) receiving, using a communication device, one or more individual data associated with one or more individuals from one or more devices at 310; analyzing, using a processing device and a machine learning chatbot, the one or more individual data, wherein the machine learning chatbot includes
   an Interactive Voice Response (IVR) module having a processor; and
   a sentiment analyzer coupled to the processor at 311;
   determining, using the processing device, one or more characteristics of the one or more individuals relevant to an employment position based on the analyzing of the one or more individual data; generating one or more unverified scores based on the one or more individual data and the one or more characteristics at 313.

The one or more individual data may include current IP addresses and information related to VPN (virtual private network). Further, the one or more individual data may include background data associated with a personal background of the one or more individuals. Further, the background data may include working experience, volunteering experience, educational background, academic standing (GPA, number of publications etc.), different measurable quotients (IQ, EQ, AQ etc), measurable skills, skill levels of the measureable skills, etc. Further, the background data may include education level, major/minor, GPA, date of birth, work experience, all certificates/test scores, etc. that they have taken previously (IELTS, TOEFL, GRE, GMAT, etc.). Further, the background data may include indication of a set of hard and soft skills the one or more individuals perceive to possess. Further, the one or more individuals may include users, clients, job applicants, candidates, test takers, applicants, etc. Further, the one or more devices may include the one or more client devices, one or more computing devices, etc.

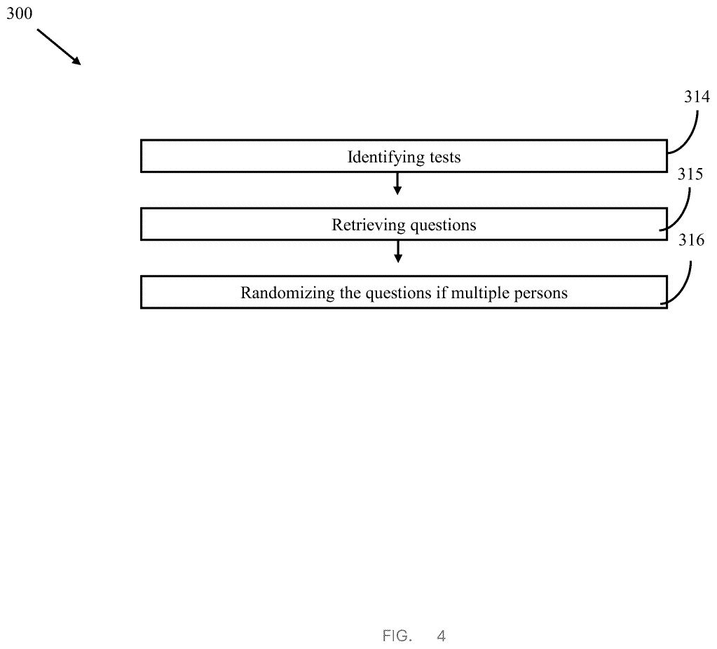

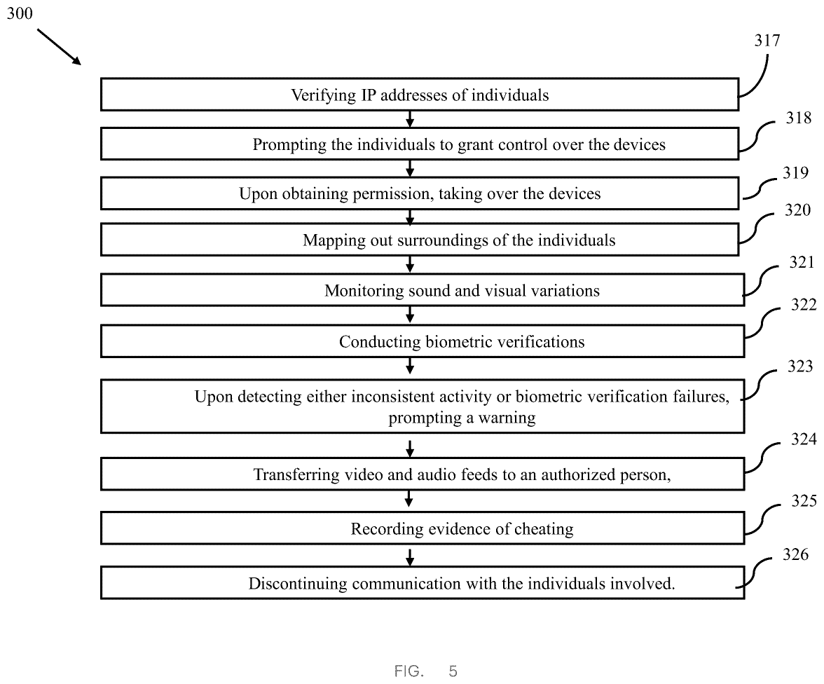

As shown in FIG. 4, the method 300 may further include a steps for Identifying Tests. In one embodiment, the method 300 may include steps of
b) identifying, using the processing device, one or more tests for verifying the one or more characteristics based on the determining of the one or more characteristics at 314;
   retrieving, using a storage device, one or more questions associated with the one or more tests based on the identifying of the one or more tests at 315; randomizing the one or more questions, using the processing device, if multiple persons are included in the one or more individuals at 316.
      The one or more characteristics may include Hard skills, Soft skills, Education, Age, Health, Intelligence (IQ), Emotional Intelligence (EQ), Resilience (AQ), Creativity (CQ), etc. Further, the one or more tests may include Hard skill tests, Soft skill tests, Education tests, Intelligence (IQ) tests, Emotional Intelligence (EQ) tests, Resilience (AQ) tests, Creativity (CQ) tests, etc.
      As shown in FIG. 5, the method 300 may further include steps for Control and

### Monitoring.

In one embodiment, the method 300 may include steps of
c) verifying IP addresses of the one or more individuals at 317;
   prompting, using the communication device, the one or more individuals via the one or more devices, to grant control over the one or more devices at 318; upon obtaining permission, taking over the one or more devices at 319; mapping out, using the communication device and the machine learning chatbot, surroundings of the one or more individuals at 320;
   monitoring, using a communication device and the machine learning chatbot, sound and visual variations proximate to one or more individuals at 321, conducting both random and scheduled biometric verifications based on individual data at 322; upon detecting either inconsistent sound or visual activity or a predetermined number of biometric verification failures, prompting a warning to the one or more individuals at 323, transferring one or more video and audio feeds from the devices controlled by the one or more individuals to an authorized person, thereby enabling the authorized person to visually monitor the activities of the one or more individuals at 324; recording evidence of cheating upon initiation by the authorized person following the detection of cheating at 325; and discontinuing communication with the one or more individuals involved at 326.

In some embodiments, the one or more devices may include one or more location sensors. Further, the one or more location sensors may be configured for generating the one or more preliminary monitoring data based on detecting a location of the one or more individuals. Further, the one or more preliminary monitoring data may include the location of the one or more individuals. Further, the one or more external sensors may include image sensors, sound sensors, motion sensors, etc. Further, the one or more external sensors may be configured for generating the one or more monitoring data based on monitoring one or more of the one or more individuals and one or more environments of the one or more individuals. Further, the monitoring of the one or more individuals may include detecting a movement, a voice, an eye movement, etc. of the one or more individuals. Further, the monitoring of the one or more environments may include detecting an envionment's sound, an environment's visual, a movement of objects, etc. of the one or more environments.

In some other embodiments, the one or more devices may include one or more internal sensors. Further, the one or more internal sensors may be configured for generating the one or more monitoring data based on monitoring one or more of a software configuration and a hardware configuration of the one or more devices. Further, the monitoring of the software configuration may include detecting one or more software executed on the one or more devices, detecting a data traffic of the one or more software, detecting a data packet of the one or more software, etc. Further, the monitoring of the hardware configuration may include detecting connecting of an external device to the one or more devices, detecting a removal of a component from the one or more devices, etc.

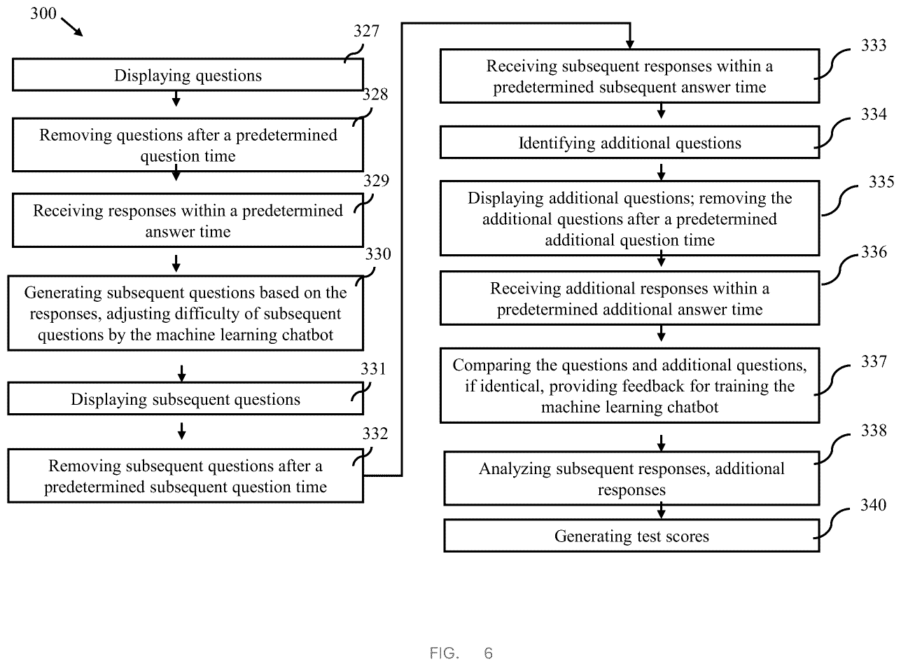

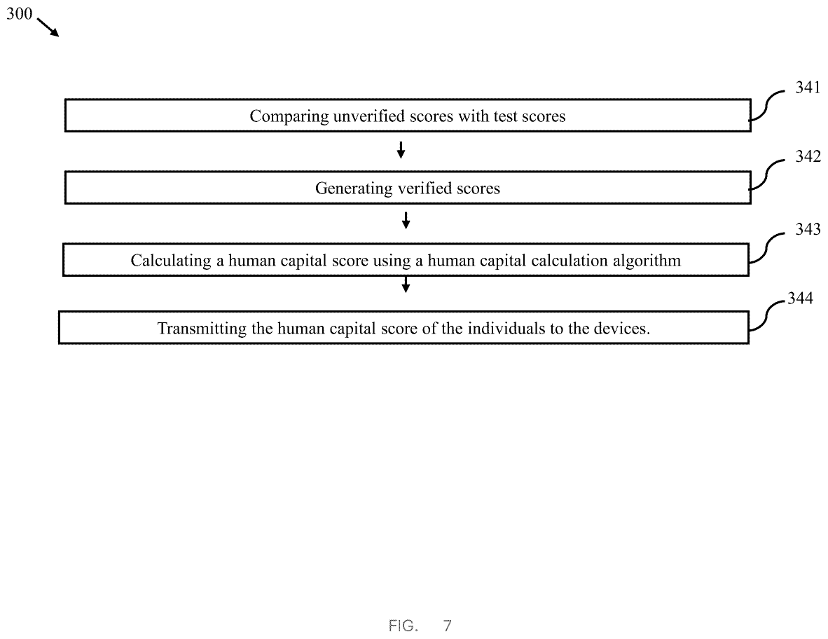

As shown in FIG. 6, the method 300 may further include steps for Test Questions.
In one embodiment, the method 300 may further include steps of:
d) displaying, using the communication device, the one or more questions on the one or more devices at 327;
   removing, using the communication device, the one or more questions from the one or more devices after a predetermined question time at 328; receiving, using the communication device, one or more responses corresponding to the one or more questions from the one or more devices within a predetermined answer time at 329; generating, using the communication device, one or more subsequent questions based on the one or more responses, adjusting difficulty of subsequent questions by the machine learning chatbot at 330; displaying, using the communication device, the one or more subsequent questions on the one or more devices at 331; removing, using the communication device, the one or more subsequent questions from the one or more devices after a predetermined subsequent question time at 332; receiving, using the communication device, one or more subsequent responses corresponding to the one or more subsequent questions from the one or more devices within a predetermined subsequent answer time at 333; identifying, using the processing device and the machine learning chatbot, one or more additional questions based on the employment position, the one or more individual data and the one or more characteristics at 334; displaying, using the communication device, the one or more additional questions on the one or more devices at 335; removing, using the communication device, the one or more additional questions from the one or more devices after a predetermined additional question time at 335; receiving, using the communication device, one or more additional responses corresponding to the one or more additional questions from the one or more devices within a predetermined additional answer time at 336; comparing the one or more questions and the one or more additional questions to identify identical questions, if identical questions are found, providing feedback for training the machine learning chatbot at 337; analyzing, using the processing device, the one or more subsequent responses and the one or more additional responses at 338; generating, using the processing device, one or more test scores corresponding to the one or more characteristics based on the analyzing of the one or more subsequent responses and the one or more additional responses at 339.
      The one or more responses may include answers to the one or more questions.
      As shown in FIG. 7, the method 300 may include steps for Generating Human Capital Scores.
      In one embodiment, the method 300 may further include steps of:
e) comparing the one or more unverified scores with the one or more test scores at 341; generating one or more verified scores at 342; calculating, using the processing device, a human capital score for the one or more individuals using a human capital calculation algorithm based on the one or more verified scores, wherein the human capital score indicates the fitness of the one or more individuals for the employment position at 343; and transmitting, using the communication device, the human capital score of the one or more individuals to the one or more devices at 344.

In some embodiments, the one or more unverified scores can be user-defined. The human capital score indicates the fitness of the one or more individuals for the employment position. Further, the human capital calculation algorithm may include “One size fits ALL” formula.

The present invention may additionally incorporate the following detailed features.

In some embodiments, the method 300 and system (that includes at the method 300) of the present invention may include the Interactive Voice Response module having a processor configured to process digits, voice, and text data.

In some embodiments, the method 300 and system of the present invention may include the sentiment analyzer configured to analyze prosodic data from the voice data, wherein the prosodic data includes at least one of intonation, tone, stress, and rhythm, to determine an emotional state of the one or more individuals.

In some embodiments, the method 300 and system of the present invention may further include a step of storing, using the storage device, the human capital score of the one or more individuals.

In some embodiments, the method 300 and system of the present invention may include the one or more verified scores having the one or more test scores that match the one or more unverified scores.

In some embodiments, the method 300 and system of the present invention may include the one or more devices that comprises at least one or more cameras and one or more microphones configured to monitor surroundings of the one or more individuals.

In some embodiments, the method 300 and system of the present invention may further include detecting VPN of the one or more individuals based on the one or more individual data.

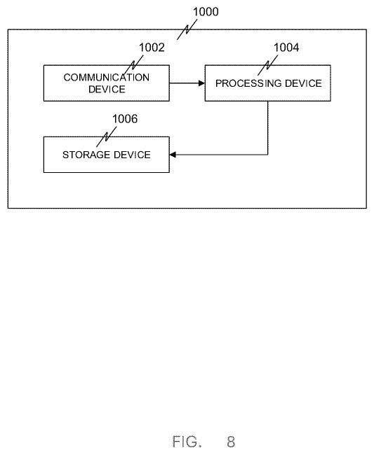

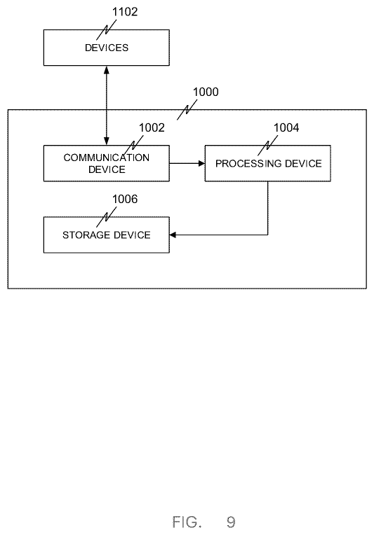

In some embodiments, the method 300 and system of the present invention may further include blocking access to one or more search engines and one or more browsers. FIG. 8 illustrates a block diagram of a system 1000 for facilitating determining fitness of individuals for an employment position, in accordance with some embodiments. Accordingly, the system 1000 may include a communication device 1002. Further, the communication device 1002 may be configured for receiving one or more individual data associated with one or more individuals from one or more devices 1102, as shown in FIG. 9. Further, the communication device 1002 may be configured for transmitting one or more questions to the one or more devices 1102. Further, the communication device 1002 may be configured for receiving one or more responses corresponding to the one or more questions from the one or more devices 1102. Further, the communication device 1002 may be configured for transmitting a human capital score of the one or more individuals to the one or more devices 1102. Further, the communication device 1002 may include a communication interface.

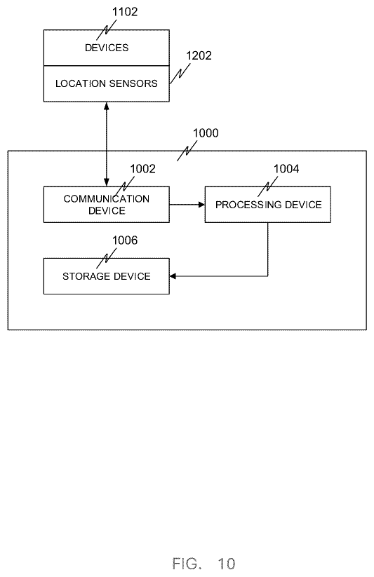

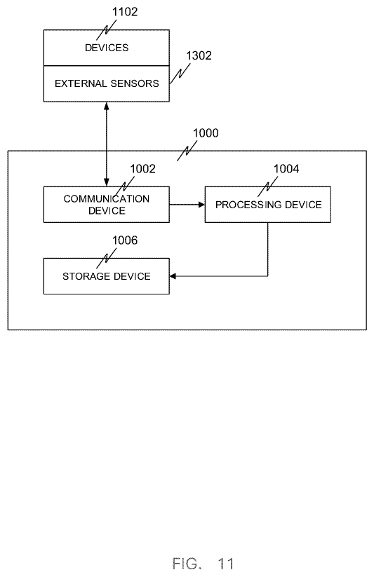

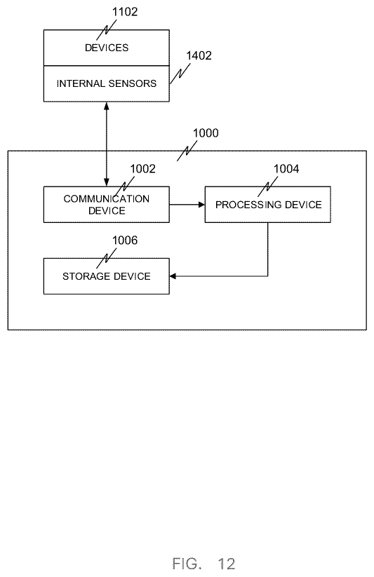

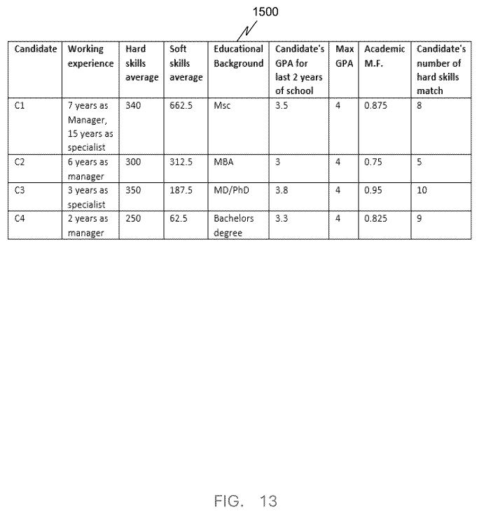

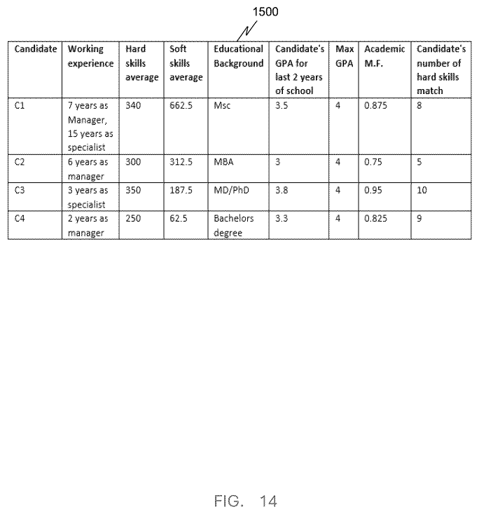

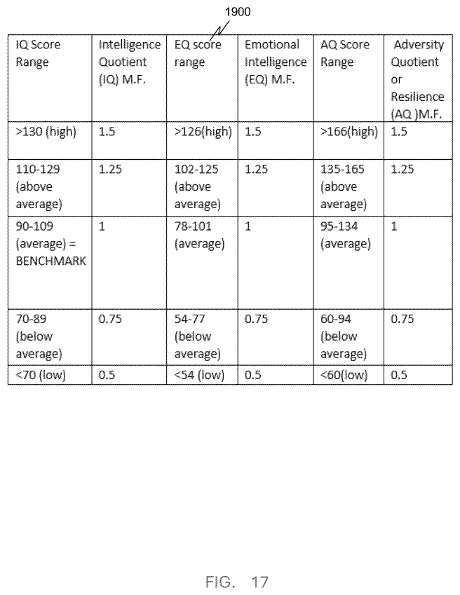

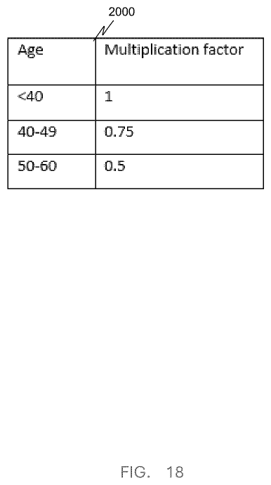

Further, the system 1000 may include a processing device 1004 communicatively coupled with the communication device 1002. Further, the processing device 1004 may be configured for analyzing the one or more individual data. Further, the processing device 1004 may be configured for determining one or more characteristics of the one or more individuals relevant to the employment position based on the analyzing of the one or more individual data. Further, the processing device 1004 may be configured for identifying one or more tests for verifying the one or more characteristics based on the determining of the one or more characteristics. Further, the processing device 1004 may be configured for analyzing the one or more responses with the one or more questions. Further, the processing device 1004 may be configured for generating one or more scores corresponding to the one or more characteristics based on the analyzing of the one or more responses. Further, the processing device 1004 may be configured for calculating the human capital score for the one or more individuals using a human capital calculation algorithm based on the one or more scores. Further, the human capital score may indicates the fitness of the one or more individuals for the employment position. Further, the processing device 1004 may include a processing unit.
Further, the system 1000 may include a storage device 1006 communicatively coupled with the processing device 1004. Further, the storage device 1006 may be configured for retrieving the one or more questions associated with the one or more tests based on the identifying of the one or more tests. Further, the storage device 1006 may be configured for storing the human capital score of the one or more individuals. Further, the storage device 1006 may include a memory.
Further, in some embodiments, the communication device 1002 may be configured for receiving a request for the determining of the fitness of the one or more individuals for the employment position from the one or more devices 1102. Further, the communication device 1002 may be configured for receiving one or more preliminary monitoring data associated with the one or more individuals from the one or more devices 1102. Further, the processing device 1004 may be further configured for analyzing the one or more preliminary monitoring data based on one or more criteria. Further, the processing device 1004 may be configured for determining an eligibility of the one or more individuals based on the analyzing of the one or more preliminary data. Further, the retrieving of the one or more questions may be further based on the eligibility of the one or more individuals.
In some embodiments, the one or more devices 1102 may include one or more location sensors 1202, as shown in FIG. 10. Further, the one or more location sensors 1202 may be configured for generating the one or more preliminary monitoring data based on detecting a location of the one or more individuals. Further, the one or more preliminary monitoring data may include the location of the one or more individuals.
Further, in some embodiments, the storage device 1006 may be configured for retrieving one or more rules associated with the one or more tests based on the identifying of the one or more tests. Further, the communication device 1002 may be configured for transmitting the one or more rules to the one or more devices 1102. Further, the communication device 1002 may be configured for receiving an acceptance to the one or more rules by the one or more individuals from the one or more devices 1102. Further, the retrieving of the one or more questions may be further based on the receiving of the acceptance.
Further, in some embodiments, the communication device 1002 may be configured for receiving one or more monitoring data associated with the one or more individuals from the one or more devices 1102. Further, the processing device 1004 may be configured for analyzing the one or more monitoring data using one or more machine learning models based on the one or more rules. Further, the processing device 1004 may be configured for determining a violation of the one or more rules based on the analyzing of the one or more monitoring data. Further, the generating of the one or more scores may be further based on the determining of the violation.
In some embodiments, the processing device 1004 may be further configured for generating a warning for the violation based on the determining of the violation. Further, the communication device 1002 may be further configured for transmitting the warning to the one or more devices 1102.
In some embodiments, the one or more devices 1102 may include one or more external sensors 1302, as shown in FIG. 11. Further, the one or more external sensors 1302 may be configured for generating the one or more monitoring data based on monitoring one or more of the one or more individuals and one or more environments of the one or more individuals.
In some embodiments, the one or more devices 1102 may include one or more internal sensors 1402, as shown in FIG. 12. Further, the one or more internal sensors 1402 may be configured for generating the one or more monitoring data based on monitoring one or more of a software configuration and a hardware configuration of the one or more devices 1102.
In some embodiments, the storage device 1006 may be further configured for retrieving one or more data associated with the employment position. Further, the processing device 1004 may be further configured for analyzing the one or more data. Further, the determining of the one or more characteristics may be further based on the analyzing of the one or more data.
Further, in some embodiments, the one or more characteristics may include two or more characteristics. Further, the communication device 1002 may be configured for transmitting the two or more characteristics to the one or more devices 1102. Further, the communication device 1002 may be configured for receiving two or more unverified scores corresponding to the two or more characteristics from the one or more devices 1102. Further, the two or more unverified scores may be user-defined. Further, the processing device 1004 may be configured for analyzing the two or more unverified scores and the one or more scores. Further, the processing device 1004 may be configured for identifying one or more acceptable scores from the two or more unverified scores for one of the two or more characteristics based on the analyzing of the two or more unverified scores and the one or more scores. Further, the calculating of the human capital score may be further based on the one or more acceptable scores.
FIG. 9 illustrates a block diagram of the system 1000, in accordance with some embodiments.
FIG. 10 illustrates a block diagram of the system 1000, in accordance with some embodiments.
FIG. 11 illustrates a block diagram of the system 1000, in accordance with some embodiments.
FIG. 12 illustrates a block diagram of the system 1000, in accordance with some embodiments.
FIG. 13 is a table 1500 illustrating attributes of individuals (candidates) for facilitating determining fitness of individuals for an employment position, in accordance with some embodiments.
FIG. 14 is a continuation of the table 1500 illustrating the attributes of the individuals (candidates) for facilitating the determining of the fitness of the individuals for the employment position, in accordance with some embodiments.
FIG. 15 is a table 1700 illustrating work experience scores of individuals (candidates) for facilitating determining fitness of individuals for an employment position, in accordance with some embodiments.
FIG. 16 is a table 1800 illustrating educational background scores of individuals (candidates) for facilitating determining fitness of individuals for an employment position, in accordance with some embodiments.
FIG. 17 is a table 1900 illustrating quotient scores of individuals (candidates) for facilitating determining fitness of individuals for an employment position, in accordance with some embodiments.
FIG. 18 is a table 2000 illustrating age multiplication factors of individuals (candidates) for facilitating determining fitness of individuals for an employment position, in accordance with some embodiments.

Although the present disclosure has been explained in relation to its preferred embodiment, it is to be understood that many other possible modifications and variations can be made without departing from the spirit and scope of the disclosure.

What is claimed is:

1. A method comprising:
a) receiving, using a communication device, one or more individual data associated with one or more individuals from one or more devices; analyzing, using a processing device and a machine learning chatbot, the one or more individual data, wherein the machine learning chatbot includes
an Interactive Voice Response (IVR) module having a processor; and
a sentiment analyzer coupled to the processor;
determining, using the processing device, one or more characteristics of the one or more individuals relevant to an employment position based on the analyzing of the one or more individual data; generating one or more unverified scores based on the one or more individual data and the one or more characteristics;
b) identifying, using the processing device, one or more tests for verifying the one or more characteristics based on the determining of the one or more characteristics; retrieving, using a storage device, one or more questions associated with the one or more tests based on the identifying of the one or more tests; randomizing the one or more questions, using the processing device, if multiple persons are included in the one or more individuals;
c) verifying IP addresses of the one or more individuals; prompting, using the communication device, the one or more individuals via the one or more devices, to grant control over the one or more devices; upon obtaining permission, taking over the one or more devices; mapping out, using the communication device and the machine learning chatbot, surroundings of the one or more individuals; monitoring, using a communication device and the machine learning chatbot, sound and visual variations proximate to one or more individuals, conducting both random and scheduled biometric verifications based on individual data; upon detecting either inconsistent sound or visual activity or a predetermined number of biometric verification failures, prompting a warning to the one or more individuals, transferring one or more video and audio feeds from the devices controlled by the one or more individuals to an authorized person, thereby enabling the authorized person to visually monitor the activities of the one or more individuals; recording evidence of cheating upon initiation by the authorized person following the detection of cheating; and discontinuing communication with the one or more individuals involved;
d) displaying, using the communication device, the one or more questions on the one or more devices; removing, using the communication device, the one or more questions from the one or more devices after a predetermined question time; receiving, using the communication device, one or more responses corresponding to the one or more questions from the one or more devices within a predetermined answer time; generating, using the communication device, one or more subsequent questions based on the one or more responses, adjusting difficulty of subsequent questions by the machine learning chatbot; displaying, using the communication device, the one or more subsequent questions on the one or more devices; removing, using the communication device, the one or more subsequent questions from the one or more devices after a predetermined subsequent question time; receiving, using the communication device, one or more subsequent responses corresponding to the one or more subsequent questions from the one or more devices within a predetermined subsequent answer time; identifying, using the processing device and the machine learning chatbot, one or more additional questions based on the employment position, the one or more individual data and the one or more characteristics; displaying, using the communication device, the one or more additional questions on the one or more devices; removing, using the communication device, the one or more additional questions from the one or more devices after a predetermined additional question time; receiving, using the communication device, one or more additional responses corresponding to the one or more additional questions from the one or more devices within a predetermined additional answer time; comparing the one or more questions and the one or more additional questions to identify identical questions, if identical questions are found, providing feedback for training the machine learning chatbot; analyzing, using the processing device, the one or more subsequent responses and the one or more additional responses; generating, using the processing device, one or more test scores corresponding to the one or more characteristics based on the analyzing of the one or more subsequent responses and the one or more additional responses;
e) comparing the one or more unverified scores with the one or more test scores; generating one or more verified scores; calculating, using the processing device, a human capital score for the one or more individuals using a human capital calculation algorithm based on the one or more verified scores, wherein the human capital score indicates the fitness of the one or more individuals for the employment position; and transmitting, using the communication device, the human capital score of the one or more individuals to the one or more devices.

2. The method as claimed in claim 1, wherein the Interactive Voice Response module having a processor is configured to process digits, voice, and text data.

3. The method as claimed in claim 2, wherein the sentiment analyzer is configured to analyze prosodic data from the voice data, wherein the prosodic data includes at least one of intonation, tone, stress, and rhythm, to determine an emotional state of the one or more individuals.

4. The method as claimed in claim 1, wherein the method further comprising a step of storing, using the storage device, the human capital score of the one or more individuals.

5. The method as claimed in claim 1, wherein the one or more verified scores is the one or more test scores that match the one or more unverified scores.

6. The method as claimed in claim 1, wherein the one or more devices comprises at least one or more cameras and one or more microphones configured to monitor surroundings of the one or more individuals.

7. The method as claimed in claim 1, wherein the the method further comprises detecting VPN of the one or more individuals based on the one or more individual data.

8. The method as claimed in claim 1, wherein the the method further comprises blocking access to one or more search engines and one or more browsers.

9. A system, comprising:
memory storing instructions; and
a processor that executes the instructions to perform operations comprising:
f) analyzing one or more individual data by a machine learning chatbot that includes:
an Interactive Voice Response (IVR) module having a processor; and
a sentiment analyzer coupled to the processor;
determining one or more characteristics of one or more individuals;
generating one or more unverified scores;
g) retrieving one or more questions;
h) prompting the one or more individuals to grant control over the one or more devices; upon obtaining permission, taking over the one or more devices including location sensors, internal sensors, and external sensors; mapping out surroundings of the one or more individuals by using machine learning chatbot; monitoring sound and visual variations proximate to one or more individuals; determining cheating activity by using machine learning chatbot; recording evidence of cheating upon initiation by an authorized person following the detection of cheating; and discontinuing communication with the one or more individuals involved;
i) presenting the one or more questions to the one or more individuals; receiving one or more responses from the the one or more individuals; generating one or more test scores; and
j) generating one or more verified scores; calculating a human capital score for the one or more individuals using a human capital calculation algorithm based on the one or more verified scores.

10. The system as claimed in claim 9, wherein the Interactive Voice Response module having a processor is configured to process digits, voice, and text data.

11. The system as claimed in claim 10, wherein the sentiment analyzer is configured to analyze prosodic data from the voice data, wherein the prosodic data includes at least one of intonation, tone, stress, and rhythm, to determine an emotional state of the one or more individuals.

12. The system as claimed in claim 9, wherein the operations further comprising storing the human capital score of the one or more individuals.

13. The system as claimed in claim 9, wherein the one or more verified scores is the one or more test scores that match the one or more unverified scores.

14. The system as claimed in claim 9, wherein the one or more devices comprises at least one or more cameras and one or more microphones configured to monitor surroundings of the one or more individuals.

15. The system as claimed in claim 9, wherein the the operations further comprises verifying IP addresses of the one or more individuals; and
detecting VPN of the one or more individuals based on the one or more individual data.

16. The system as claimed in claim 9, wherein the the operations further comprises blocking access to one or more search engines and one or more browsers.

17. A non-transitory computer readable storage medium storing instructions which when executed by a processor cause the processor to perform operations comprising:
k) receiving, using a communication device, one or more individual data associated with one or more individuals from one or more devices; analyzing, using a processing device and a machine learning chatbot, the one or more individual data, wherein the machine learning chatbot includes
an Interactive Voice Response (IVR) module having a processor; and
a sentiment analyzer coupled to the processor;
determining, using the processing device, one or more characteristics of the one or more individuals relevant to an employment position based on the analyzing of the one or more individual data; generating one or more unverified scores based on the one or more individual data and the one or more characteristics;
l) identifying, using the processing device, one or more tests for verifying the one or more characteristics based on the determining of the one or more characteristics; retrieving, using a storage device, one or more questions associated with the one or more tests based on the identifying of the one or more tests; randomizing the one or more questions, using the processing device, if multiple persons are included in the one or more individuals;
m) verifying IP addresses of the one or more individuals; detecting VPN of the one or more individuals based on the one or more individual data; blocking access to one or more search engines and one or more browsers; prompting, using the communication device, the one or more individuals via the one or more devices, to grant control over the one or more devices; upon obtaining permission, taking over the one or more devices that include at least one or more cameras and one or more microphones configured to monitor surroundings of the one or more individuals; mapping out, using the communication device and the machine learning chatbot, surroundings of the one or more individuals using the at least one or more cameras; monitoring, using a communication device and the machine learning chatbot, sound and visual variations proximate to one or more individuals, conducting both random and scheduled biometric verifications based on individual data; upon detecting either inconsistent sound or visual activity or a predetermined number of biometric verification failures, prompting a warning to the one or more individuals, transferring one or more video and audio feeds from the devices controlled by the one or more individuals to an authorized person, thereby enabling the authorized person to visually monitor the activities of the one or more individuals; recording evidence of cheating upon initiation by the authorized person following the detection of cheating; and discontinuing communication with the one or more individuals involved;
n) displaying, using the communication device, the one or more questions on the one or more devices; removing, using the communication device, the one or more questions from the one or more devices after a predetermined question time; receiving, using the communication device, one or more responses corresponding to the one or more questions from the one or more devices within a predetermined answer time; generating, using the communication device, one or more subsequent questions based on the one or more responses, adjusting difficulty of subsequent questions by the machine learning chatbot; displaying, using the communication device, the one or more subsequent questions on the one or more devices; removing, using the communication device, the one or more subsequent questions from the one or more devices after a predetermined subsequent question time; receiving, using the communication device, one or more subsequent responses corresponding to the one or more subsequent questions from the one or more devices within a predetermined subsequent answer time; identifying, using the processing device and the machine learning chatbot, one or more additional questions based on the employment position, the one or more individual data and the one or more characteristics; displaying, using the communication device, the one or more additional questions on the one or more devices; removing, using the communication device, the one or more additional questions from the one or more devices after a predetermined additional question time; receiving, using the communication device, one or more additional responses corresponding to the one or more additional questions from the one or more devices within a predetermined additional answer time; comparing the one or more questions and the one or more additional questions to identify identical questions, if identical questions are found, providing feedback for training the machine learning chatbot; analyzing, using the processing device, the one or more subsequent responses and the one or more additional responses; generating, using the processing device, one or more test scores corresponding to the one or more characteristics based on the analyzing of the one or more subsequent responses and the one or more additional responses;
o) comparing the one or more unverified scores with the one or more test scores; generating one or more verified scores, wherein the one or more verified scores is the one or more test scores that match the one or more unverified scores; calculating, using the processing device, a human capital score for the one or more individuals using a human capital calculation algorithm based on the one or more verified scores, wherein the human capital score indicates the fitness of the one or more individuals for the employment position; and transmitting, using the communication device, the human capital score of the one or more individuals to the one or more devices.

18. The non-transitory computer readable storage medium as claimed in claim 17, wherein the Interactive Voice Response module having a processor is configured to process digits, voice, and text data.

19. The non-transitory computer readable storage medium as claimed in claim 18, wherein the sentiment analyzer is configured to analyze prosodic data from the voice data, wherein the prosodic data includes at least one of intonation, tone, stress, and rhythm, to determine an emotional state of the one or more individuals.

20. The non-transitory computer readable storage medium as claimed in claim 17, wherein the operations further comprising storing, using the storage device, the human capital score of the one or more individuals.
